(19) United States
(12) Patent Application Publication
(10) Pub. No.: US 2008/0124385 A1
(43) Pub. Date: May 29, 2008

(54) Methods for Transmembrane Treatment and Prevention of Otitis Media

(75) Inventor: William R. Campbell, Jamestown, NC (US)

Correspondence Address: DLA PIPER US LLP, 4365 EXECUTIVE DRIVE, SUITE 1100, SAN DIEGO, CA, 92121-2133, US

(73) Assignee: Piedmont Pharmaceuticals,LLC.

(21) Appl. No.: 11/661,169

(22) PCT Filed: Sep. 2, 2005
(86) PCT No.: PCT/US05/31531

§ 371 Date: Jan. 15, 2008

Related U.S. Application Data
(60) Provisional application No. 60/607,175, filed on Sep. 3, 2004.
(60) Provisional application No. 60/649,926, filed on Feb. 3, 2005.

(51) Int. Cl. A61K 9/127; A61K 31/5383; A61K 31/42; A61K 31/397; A61K 31/522

(52) U.S. Cl. 424/450; 514/230.2; 514/380; 514/210.05; 514/263.37

(57) ABSTRACT

Methods for treating and preventing middle ear infections by transmembrane administration of medicament-containing transmembrane carrier compositions, such as liposomes and other lipid vesicles, to the tympanic membrane. Medicaments useful for treating pain, inflammation or infection in the outer ear may be co-administered. If utilized for transmembrane administration, the liposomes or other lipid vesicles will usually not be sterically stabilized. The medicaments delivered according to the methods of the invention include antibiotic, anti-viral, anti-fungal and anti-inflammatory agents that are useful in treatment and/or prophylaxis of middle ear infections and their sequelae.

### FIELD OF THE INVENTION

The present invention relates to non-invasive methods for treating otitis media (middle ear infection). More particularly, the invention relates to methods for administering medicament useful in treating otitis media to the middle ear by delivery thereof across the tympanic membrane (eardrum).

### BACKGROUND

Millions of children are affected each year with otitis media; i.e., infection of the middle ear. Although adults are also susceptible to middle ear infections, children are particularly at risk, because their relatively short auditory canals can more easily be closed by inflammation. Fluid can then become trapped behind the tympanic membrane (eardrum), which can cause severe pain as well as provide microbes with an inviting environment in which to reproduce.

The tympanic membrane is a formidable barrier against introduction of drugs into the middle ear, and so antibiotics prescribed to treat middle ear infections are nearly always taken orally. However, a variety of bacteria and viruses can be responsible for causing middle ear infections, and it is frequently not possible to distinguish which is the cause of a particular infection, or whether it is susceptible to treatment with oral antibiotics. Further, the impact of orally administered antibiotics on the middle ear may be diluted by the systemic distribution of the drug, which may also place the patient at risk for side effects associated with systemic delivery (e.g., yeast infections in female patients).

Children who suffer from repeated infections may require surgery to relieve the fluid pressure on the tympanic membrane. In more severe cases, drainage tubes may be placed within the tympanic membrane. The tubes themselves don't prevent reoccurrences of infection (to the contrary, they can serve as conduits for entry of additional pathogens into the middle ear), but they can relieve pressure and reduce the extent to which fluid becomes trapped behind the eardrum. The tubes also offer a potential conduit for antibiotics to be introduced directly into the middle ear; e.g., by applying antibiotic drops and allowing them to flow into the drainage tube. However, this method is both invasive and painful, suggesting a strong need for an alternative route for introducing antibiotics into the middle ear.

### SUMMARY OF THE INVENTION

Surprisingly, it has been found that medicaments can be introduced directly into the middle ear by transmembrane delivery. According to the invention, the medicament is supplied as an active ingredient of a transmembrane carrier composition applied to the tympanic membrane (eardrum), such as a lipid-based emulsion, lipid vesicle, liposomes, liosomes, micelles, transferomes and polymeric carriers capable of delivering an agent across the tympanic membrane.

Preferred medicaments are those useful in the treatment or prevention of otitis media (middle ear infection) and its sequelae. The invention is particularly well-suited to the delivery of medicaments such as antibiotics or anti-viral agents (depending on the source of the infection present), anti-fungal agents, and anti-inflammatory agents or other painkillers. For prevention of chronically recurring middle ear infections, the methods of the invention may also be utilized between active infections to deliver prophylactic agents to the middle ear.

The summary of the invention described above is not limiting and other features and advantages of the invention will be apparent from the following detailed description of the preferred embodiments, as well as from the claims.

### DETAILED DESCRIPTION OF THE INVENTION

### A. Methods for Transmembrane Treatment of Otitis Media

The present invention provides methods for treating and preventing otitis media through administration of medicaments useful in prophylaxis or treatment of middle ear infections and their sequelae in a transmembrane carrier composition. The invention derives from the surprising discovery that, in an appropriate carrier, medicaments can be delivered across the tympanic membrane, without puncturing the membrane (e.g., by insertion of tubes or injection).

By “transmembrane administration” is meant that a transmembrane carrier composition of the invention capable of crossing the tympanic membrane is applied on the outer ear side of the tympanic membrane to deliver a medicament to the middle ear. Thus, the invention provides methods for preventing and/or treating infections of the middle ear and their sequelae by transmembrane administration of a medicament to the tympanic membrane of the affected individual.

Transmembrane administration is achieved via, for example, applying the transmembrane carrier composition of the invention to the tympanic membrane via any medically acceptable means for application of a pharmaceutical composition to the tympanic membrane; e.g., by applying the carrier composition to the membrane by insertion of a needleless syringe or dropper into the auditory canal. Administration is repeated as required to achieve the therapeutically effective dosage level for the antibiotic compound given; for example, 5-10 drops of a transmembrane carrier composition consisting of 0.3% w/w of antibiotic could be delivered twice a day to treat otitis media in an affected child.

Those of ordinary skill in the art will be familiar with, and readily able to select, dosing regimens suitable for following to treat a particular infection. The dosing regimen selected will be in accord with established clinical protocols for delivery and use of the particular carrier and medicaments provided according to the invention. In one embodiment the medicament is provided at a concentration in a lipid-based carrier of at least about 0.3% w/w.

The compositions are preferably administered with the transmembrane carrier composition itself as a carrier, but in various embodiments the transmembrane carrier may be administered in a carrier gel or other suitable carrier.

### B. Lipid-Based Carriers For Use In The Invention

Although the invention shall not be limited by any theory as to the mechanism of action for such delivery, it is presently believed that a carrier suitable for transmembrane delivery of a medicament is one that is capable of interaction (e.g., Van der Waals interaction) with, and possibly also entry into, lipid-rich channels in the tympanic membrane. Hence, presently preferred transmembrane carriers are those that are lipid-based, such as lipid emulsions (including microemulsions and oil-in-water emulsions), as well as lipid vesicles, such as liposomes, liosomes, micelles and transfersomes (ultraflexible lipid vesicles). Phospholipid-based formulations are presently preferred, especially for the non-vesicular formulations useful in the invention.

Again without limiting the invention by any theory of its mechanism of action, it is also observed that transmembrane delivery is most efficient in an acute phase of infection, wherein the tympanic membrane bulges outwardly (i.e., into the outer ear) due to pressure build-up in the middle ear, a symptomatic hallmark of acute otitis media infection. Bulging indicates that fluid has become trapped behind the membrane. The introduction of the lipid-based carriers of the invention onto the opposite side of the membrane may create an osmotic pressure differential that facilitates transmembrane transfer of medicament, either from or together with the lipid-based carrier.

Most preferably, lipid vesicle transmembrane compositions are flexible, in that they do not include a steric stabilizing component, such as cholesterol (although sterically stable vesicles may be used to co-administer medicaments into the outer ear, as further discussed elsewhere below). Further, the medicament delivered according to the invention is preferably carried in a lipid phase (e.g., in the lipid bilayer of a liposome) rather than in an aqueous phase (e.g., in the core of a liposome). Thus, lipid-soluble medicaments (which can generally be provided at a higher concentration in the lipid layer of a vesicle than a water-soluble medicament dispersed in an aqueous phase can be) are preferred, though not required, for use in the invention.

Methods for preparing lipid emulsions and vesicles are well-known in the art, and so will only be briefly outlined here with respect to the most presently preferred embodiment of transmembrane carrier composition for use in the invention, a liposome prepared without a steric stabilizer, and with little or no addition of a viscocity-enhancing agent.

By “liposome” is meant a spherical vesicle bounded by an ordered lipid bilayer and enclosing an aqueous phase. The lipid bilayer of liposomes is usually made of natural or synthetic phospholipids, but can also be made of non-phospholipids. The lipid bilayer of liposomes is an ordered bilayer, meaning that the molecular “head” and “tail” structures of the lipids are lined up next to one another.

Liposomes utilized in the present invention can be unilamellar (having one lipid bilayer) or more preferably are multilamellar. Liposomes that are “multilamellar” have multiple layers or membranes. This type of liposome has layers of lipid bilayers with an aqueous fluid spaced in between the lipid bilayers. Multilamellar liposomes have at least two layers of lipids.

Preferred liposomes are those described herein, and in co-pending and commonly owned U.S. patent application Ser. No. 10/366,584, filed on Feb. 12, 2003, the disclosure of which is incorporated herein in its entirety, by this reference. However, those of ordinary skill in the art will recognize that other formulations of liposomes may be utilized, including phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine. Such lipids will also be useful in non-vesicular transmembrane carrier compositions of the invention.

The size of liposomes and lipid vesicles utilized in the present invention, if any, may be variable, but such vesicles are preferably of uniform size in each batch preparation. The liposomes may be up to 20 μm, 25 μm, or even 30 μm. But in preferred embodiments about 95% of the liposomes will be from about 0.5 μm to about 10 μm in diameter. In one embodiment, at least 80% of liposomes in a preferred composition manufactured according to the methods described herein are from about 0.5 μm to about 5 μm. In this respect, the term “about” encompasses a range of 5% upwards or downwards from the stated value. The actual diameters of the liposomes will be a function of the cooling curve followed and the length and vigor of stirring or vortex hydration, when those processes are used in the manufacture of the liposomes. In still other embodiments, the liposomes can be multilamellar liposomes where a single larger liposome encapsulates one or more smaller liposomes.

Conventional liposomes manufactured according to means well known in the art may be used in the invention, but the preferred liposomes of the present invention do not contain a lipid soluble preservative as found in liposomes of the prior art (see, e.g., U.S. Pat. Nos. 4,761,288 and 4,897,269, both to Mezei, are both hereby incorporated by reference in their entirety). Rather, as described in co-pending U.S. patent application Ser. No. 10/366,584 (incorporated herein in its entirety), the liposomes of the present invention utilize a water-soluble preservative that can function as an antimicrobial, which is preferably a benzethonium salt, such as benzethonium chloride.

In this respect, “preservative” refers to an ingredient added to the transmembrane carrier composition that prevents microbes from substantially growing and multiplying in the formulation. Further, by “water soluble” is meant that the ingredient has a solubility in water in excess of 100 μg/ml (or 0.01%) in water. In other embodiments, the ingredient can have a solubility in water in excess of 1 mg/ml (0.1%).

However, other water soluble preservatives will also find use in the invention, such as benzoic acid, and benzylkonium salts such as benzylkonium chloride. It was discovered unexpectedly that the choice of the preservative is important in order to achieve stable liposomes, as lipid-soluble preservatives can weaken and destabilize the liposomes' structure due to microbial growth, leading to an unstable composition with low viscosity. Other water-soluble preservatives can be used and are advantageously selected to be active at the pH of the composition.

In a preferred embodiment of the invention, the liposomes utilized also contain vitamin E as a lipid-soluble anti-oxidant. Anti-oxidants act as free radical scavengers, facilitating the achievement of maximum stability for the liposomes. Methylcellulose or other viscosity enhancing agents are included in transmembrane carrier compositions that are to be applied to the skin in order to achieve sufficient viscosity and avoid a fluid composition. In a most preferred embodiment, the present compositions include vitamin E as an anti-oxidant and includes less than 2% w/w or less than 1.5% or less than 1.0%, or less than 0.5%, or less than 0.25% of a viscosity enhancing agent.

More preferably, the compositions do not include any methylcellulose or any other viscosity enhancing agents, which allows for optimal transmembrane penetration of the active medicament compound. In one embodiment at least 50% of the Vitamin E is present in the lipid bilayers of the liposomes. In other embodiments, at least 70% or 80% or 90% or 95% of the vitamin E is present in the lipid layers of the liposomes.

By “viscosity enhancing agents” is meant an agent that is added to the composition to increase the viscosity. A viscosity enhancing agent will increase the viscosity of the composition by at least 10,000 centipoise at 25° C. Viscosity enhancing agents include, but are not limited to, methyl cellulose, alginic acid, gelatin, acacia (gum Arabic) carbomer, and cetostearyl alcohol. Phospholipids are not considered viscosity enhancing agents within this definition. The viscosity enhancing agent will increase the viscosity by at least 10,000 centipoise versus its absence, and in other embodiments can increase the viscosity by 20,000 or 30,000 centipoise (to as high as 40,000 or 50,000 centipoise) versus its absence in the composition.

Thus, in various embodiments the preferred transmembrane carrier compositions of the present invention contain less than 2% w/w or less than 1%, less than 0.5%, or even 0% of viscosity enhancing agents. For example, in various embodiments the compositions contain less than these quantities of organic or inorganic salts, such as salts of hydrochloric acid, nitric acid, sulfuric acid, phosphoric acid, carbonic acid, hydrobromic acid, or hydroiodic acid. The compositions also preferably contain less than 2% w/w or less than 1% or even 0% of potassium bromide, potassium chloride, potassium dihydrogen phosphate, dipotassium hydrogen phosphate, potassium sulfate, potassium iodide, potassium nitrate, lithium bromide, lithium chloride, lithium iodide, lithium nitrate, lithium sulfate, ammonium bromide, ammonium chloride, ammonium carbonate, ammonium hydrogen carbonate, ammonium dihydrogen phosphate, diammonium hydrogen phosphate, ammonium iodide, ammonium nitrate, ammonium sulfate, sodium bromide, sodium carbonate, sodium chloride, sodium hydrogen carbonate, sodium dihydrogen phosphate, disodium hydrogen phosphate, sodium nitrate, sodium phosphate, and sodium sulfate.

Other salts that preferably are present in the transmembrane carrier compositions at less than 2% w/w or less than 1% or even 0% include alkanolamine chloride, sulfate, phosphate, salts of benzoic acid, acetic acid, salicyclic acid, oxalic acid phthalic acid, gluconic acid, 1-naphthalenesulfonic acid, 2-naphthalenesulfonic acid, tartaric acid, maleic acid, malonic acid, succinic acid, fumaric acid, propionic acid, ascorbic acid, mandelic acid, malic acid, citric acid, triethanolammonium chloride, triethanolammonium dihydrogen phosphate, triethanolammonium sulfate, sodium benzoate, potassium benzoate, ammonium benzoate, sodium acetate, potassium acetate, ammonium acetate, sodium salicylate, potassium salicylate, ammonium salicylate, sodium oxalate, potassium oxalate, ammonium oxalate, sodium phthalate, potassium phthalate, ammonium phthalate, sodium gluconate, potassium gluconate, ammonium gluconate, ammonium 1-naphthalenesulfonate, potassium 2-naphthalenesulfonate, ammonium 2-naphthalenesulfonate, sodium 2-naphthalenesulfonate, potassium tartarate, sodium maleate, potassium maleate, sodium malonate, sodium succinate, sodium fumarate, sodium propionate, triethanolammonium propionate, sodium ascorbate, triethanolammonium ascorbate, potassium ascorbate, sodium mandelate, sodium malate, sodium citrate, potassium citrate, and triethanolammonium citrate.

In various embodiments, transmembrane carrier compositions useful in the invention have a viscosity of at least 10,000 centipoise, or at least 20,000 centipoise, or at least 30,000 centipoise, or at least 40,000 centipoise, or at least 50,000 centipoise, or at least 60,000 centipoise, or at least 70,000 centipoise, all at 58° C., without the presence of any methyl-cellulose or other viscosity enhancing agents. Because the methylcellulose and other viscosity enhancing agents are not present in the formulations, transmembrane penetration is increased substantially. In one embodiment, oleyl alcohol may be added to enhance the transmembrane penetration of the medicament that is in the composition but is present outside the liposomes.

Without wanting to be bound by any particular theory, it is believed that it is the combination of the water soluble preservative and the lipid soluble anti-oxidant that provides stability to these particular liposomes. This enables the liposomes to be stable and also have a high viscosity. The high viscosity is possible even though the transmembrane carrier composition contains very little or no viscosity enhancing agents. It is believed viscosity enhancing agents impair the movement of active compound across the skin. The present compositions offer the superior property of a maximum degree of skin penetration in the administration of the active while retaining a sufficient viscosity.

If the liposome is a phospholipid based vesicle, a preferred lipid will be phospholipon 90H, which is obtained and purified from soy lecithin and has the chemical name 1,2-dia-cyl-5N-glycero-3-phosphatidyl choline. It is minimum 90% phophatidyl choline and is fully hydrogenated. But the person of ordinary skill will realize that other lipids may also be used in the present invention. For example, the phosphatidylcholine can be of lower purity, or can contain other lipids or carrier materials such as, for example, propylene glycol/ethanol, medium chain triglycerides, oil/ethanol, phosphatidic acid, cholesterol, and phosphatidylinositol. The phospholipid may be any natural or synthetic phospholipid, for example phosphatidylethanolamine, phosphatidylserine, phosphatidylinositol, phosphatidylglycerol, phosphatidic acid, lysophospholipids, egg or soybean phospholipid or a combination thereof. The phospholipid may be salted or desalted, hydrogenated or partially hydrogenated, natural, synthetic, or semisynthetic. Examples of commercially available phospholipids include but are not limited to egg phospholipids P123 (Pfanstiehl, Waukegen, Ill.), Lipoid E80 (Lipoid, Ludwigshafen, Germany); and the hydrogenated soy phospholipids Phospholipon 80H®, 80G®, 90H® and 100H® (Nattermann, Munich, Germany) and 99% pure soy phosphatidyl choline (Avanti Polar Lipids, Alabaster, Ala.).

Optionally, dehydrated alcohol and propylene glycol can be used as co-solvents of the lipid phase, and vitamin E acetate can be included as an anti-oxidant. In various embodiments, other lipids or lipid-like substances are used in the invention, such as ceramides, lecithins, phosphatidyl ethanolamines, phosphatidyl serines, cardiolipins, trilinoleins and like compounds. Nonphospholipids may also be used in the present invention. For example, nonphospholipid materials that may be useful include lipid vesicle forming polyoxyethylene fatty esters, polyoxyethylene fatty acid ethers, diethanolamines, long-chain acyl amides, long-chain acyl amino acid amides, long-chain acyl amides, polyoxyethylene sorbitan oleates, polyoxyethylene glycerol monostearates, glycerol monostearates, and mixtures, analogs, and derivatives thereof. The vesicles may also include a steroid, and a charge producing agent. Preferred steroids include cholesterol, hydrocortisone, and analogs, derivatives, and mixtures thereof. Preferred negative charge producing materials are oleic acid, dicetyl phosphate, palmitic acid, cetyl sulphate, retinoic acid, phosphatidic acid, phosphatidyl serine, and mixtures thereof. In order to provide a net positive charge to the vesicles when desired, long chain amines, e.g., stearyl amines or oleyl amines, long chain pyridinium compounds, e.g., cetyl pyridinium chloride, quaternary ammonium compounds, or mixtures of these can be used, so long as the lipid vesicle can carry sufficient quantities of the aqueous phase.

Other liposomal formulations, including non-phospholipid lipsomes, may be utilized in the invention. For general reference, the multiphase liposomal drug delivery system disclosed in U.S. Pat. No. 4,761,288, issued Aug. 2, 1988 to Mezei (the disclosure of which is incorporated herein for ease of reference), is an exemplary representative of liposome compositions that may be utilized in the invention. For use as a transmembrane carrier, modification of the liposome (or other lipid vesicle utilized in the invention) to sterically stabilize the vesicle, or to provide for targeting, or to provide the vesicle (or other lipid-based carrier utilized) with slow release properties, may interfere with the transmembrane activity of the composition, and is therefore not preferred.

Transmembrane carrier compositions preferred for use in the invention are “stable” meaning that they can be stored for at least 6 months, 1 year, or 2 years without changing the chemical or physical properties of the composition.

There is no theoretical limit to the number of compounds that may be incorporated into a lipid-based carrier for use in the invention. However, as those of ordinary skill in the art are aware, encapsulation efficiency is generally greater in liposomal compositions having a relatively high lipid:water content and a lipid-soluble drug carried in a lipid phase may generally be provided in a higher concentration than a water-soluble drug carried in an aqueous phase.

For example, two or more ingredients can be encapsulated in the same vesicle, or if the active compounds are incompatible, the compounds can be encapsulated separately and the transmembrane carrier compositions combined to provide a composition with two or more indications, or that treats a single indication with multiple active compounds.

It is also possible to contemporaneously treat the middle ear and the auditory canal (e.g., to treat an infection in the former and reduce swelling in the latter) by administering a transmembrane carrier composition including a first set of one or more active compounds for treatment of the middle ear encapsulated in the vesicle, and a second set of one or more active compounds for treating the auditory canal dispersed in unencapsulated form in a surrounding water phase. The encapsulated first set of compounds will cross the tympanic membrane into the middle ear; the unencapsulated second set of compounds will not.

Co-administration may also be achieved by, for example, administering the second set of compounds in a slow release form, such as in liposomes manufactured to resist degradation. Those of ordinary skill in the art will be familiar with methods of manufacture that will accomplish this goal including, without limitation, addition of cholesterol to the lipid phase (see, e.g., U.S. Pat. No. 6,352,716, incorporated herein by reference as an illustration of a method for incorporating cholesterol into liposomes to this end) and use of viscocity-enhancing agents (such as methylcellulose) during liposome manufacture.

Such relatively insoluble lipid vesicles are less suitable for delivery of medicaments across the tympanic membrane, but can instead be expected to remain where delivered, to slowly release medicament comprising the second (or further) set of compounds into the auditory canal (e.g., to treat inflammation therein, or provide a pain killer). Such vesicles may also have disinfectant or other properties helpful in treating or controlling the rate of infection in the outer ear; e.g., if hexadecyl trimethylammonium bromide, a potent disinfectant, is utilized as a positive charge producing material within the vesicles provides a secondary advantage. In such embodiments, the vesicles act as a sustained release germicide carrier as they each deteriorate.

### C. Useful Medicaments for Treatment and Prophylaxis of Otitis Media

By “medicament” is meant any biologically active compound useful in the treatment and/or prevention of middle ear infections and their sequelae, as well as associated pain and inflammation. In this respect, therefore, particularly preferred medicaments are antibiotics useful in the treatment or prevention of middle ear infections in mammals, especially humans. Depending on the severity of the infection and its cause, such antibiotics include, without limitation, amoxicillin (and other penicillins), ciprofloxacin (and other quinolone antibiotics, such as ofloxacin), clavulanate (and other beta-lactamase inhibitors), cefaclor (and other cephalosporins, such as cefixime), azithromycin (and other macrolide antibiotics, such as clarithromycin), and sulfisoxazole (as well as other sulfa drugs, such as sulfamethoxazole). Of the antibiotics useful in the invention, ciprofloxacin is presently preferred.

Sulfisoxazole and amoxicillin are the principal antibiotics that are also accepted for use in prophylaxis of recurring middle ear infections. Broad spectrum antibiotics such as amoxicillin and ciprofloxacin are especially preferred for use in treating middle ear infections, especially in persons in whom an antibiotic-resistant infection is suspected.

Useful anti-inflammatory compounds for co-administration or use independent of antibiotic therapy include those that are sometimes less effective or well-tolerated in oral administration; e.g., non-steroidal anti-inflammatory compounds, such as naproxen, ketoprofen, celecoxib and indomethacin. Anti-viral compounds, such as acyclovir, may be administered in lieu of, or as an adjunct to, antibiotic compounds when clinically indicated, as may anti-fungal compositions. Other medicaments for use in the treating and preventing middle ear infections and their sequelae may also be administered by application of the transmembrane carrier compositions of the invention to the tympanic membrane.

In some embodiments, the transmembrane carrier compositions of the present invention contain more than one medicament. For example, CLAMOXYL® and AUGMENTIN® are both combination agent compositions for oral administration that are commonly prescribed for treatment of otitis media. Each composition contains two active antibiotic ingredients, amoxicillin and clavulanate. Transmembrane carrier compositions providing such multiple agents are particularly preferred for use in appropriate indications.

### EXAMPLE 1

### Exemplary Formulation A

This section provides an example of the starting materials for manufacturing a transmembrane carrier composition of the present invention containing ciprofloxcin. The transmembrane carrier composition comprises a liposome, and contains the following ingredients in the listed percentages w/w.

| | |
|---|---|
| PHOSPHOLIPON ® 90H | 5.00 or less |
| Alcohol, dehydrated, USP | 5.00 or less |
| Propylene Glycol, USP | 5.00 |
| Vitamin E acetate | 1.00 |
| Benzethonium chloride | 0.02 |
| Ciprofloxcin | 0.30 or as required to provide |
| | therapeutic dosage |
| Purified water | 76.98 or more |

### EXAMPLE 2

### Exemplary Method of Manufacture for Formulation A

This section provides an example of how to manufacture a transmembrane carrier composition of the present invention containing ciprofloxcin.

Aqueous Phase. The process is preferably practiced using two jacketed stainless steel vortex hydration chambers. Into the larger of the two chambers, purified water and benzethonium chloride were combined slowly to avoid the formation of foam or surface bubbles. Heat was applied to obtain 50° C.±2° C., the target temperature of the aqueous phase. The chamber was covered to prevent evaporation of water and equipped with a bottom port and valve to regulate flow of material out of the vessel.

Lipid Phase. A second stainless steel jacketed mixing vessel was utilized in close proximity to the first. In this secondary chamber dehydrated alcohol and propylene glycol were first combined slowly to avoid formation of foam or surface bubbles. An overhead mixer was started and heat applied to obtain 58±2° C. with a target of 58° C. When the solution reached the target temperature, ciprofloxacin was added and fully dissolved. PHOSPHOLIPON® 90H and vitamin E acetate were then added and combined with the lipid phase until dissolved/melted. A cover was used on the chamber to prevent evaporation of alcohol throughout the procedure.

Hydration of the Lipid Phase. Valves were opened on the bottom ports of the chamber, and the flow was regulated from both vessels. The aqueous phase and oil phase flowed and met at an in-line regulating tee, and a dispersing pump pulled the two phases together. The mixture was circulated through a 60 mesh dispersing screen to optimize the hydration of the lipid phase. The mixture was then directed to the top of the chamber and the entire process was circulated through the pump, back into the chamber for 10 minutes.

Cooling Phase. After circulation, the chamber jacket was allowed to cool with continued slow mixing until the temperature of the product was 28° C., completing the process. The combination of materials is preferably fast enough to mix thoroughly without causing formation of surface foam or bubbling. The cooling process is preferably slow, with cooling of about 6° C. per hour most preferable.

### EXAMPLE 3

### Use of Exemplary Formulations for Treatment of Otitis Media

To treat a middle ear infection and sequelae thereof in an affected individual, an medicament-containing liposome according to the present invention is administered to the patient by transmembrane administration to the tympanic membrane. The medicament is one which is useful in prophylaxis and/or treatment of middle ear infections, and is an antibiotic, anti-viral agent or pain-killing agent, such as a non-steroidal anti-inflammatory agent.

Transmembrane administration is achieved via, for example, applying the transmembrane carrier composition of the invention to the tympanic membrane with a needleless syringe or other device suitable for medical insertion into the auditory canal. Administration is repeated as required to achieve the therapeutically effective dosage level for the antibiotic compound given. Pain may be treated by administration in the same general manner of pain killing and/or anti-inflammatory containing transmembrane carrier compositions of the invention.

Based on current protocols utilized to introduce antibiotics into the middle ear through an in-situ tympanic drainage tube, a suitable regimen of dosing with the exemplary formulation described in Example 1 would be 5 drops/twice a day for a child under age 12, and 10 drops/twice a day for a child of age 12 or older.

Prophylactic treatment against recurrence of a middle ear infection may be provided in the same manner, utilizing a transmembrane carrier composition of the invention containing a prophylactically effective antibiotic or other medicament.

The invention having been fully described, its practice is illustrated by the examples below. These examples are representative, not exhaustive, of methods for practicing the invention and the results that can be obtained thereby.

### EXAMPLE 4

### Animal (Chinchilla) Model of Otitis Media

Chinchilla langer is ideally suited as an animal species for studying the efficacy of treatment for otitis media in humans. Chinchillas are small, have auditory capabilities quite similar to those of humans, have a cochlea with membranous architecture similar to the human cochlea, do not manifest presbycusis in long-term studies, and lack susceptibility to naturally occurring middle ear infections, which are common to the guinea pig and rabbit. See, e.g., Hajek D M, Yuan Z, Quartey M K, Giebink G S., Otitis Media: The Chinchilla Model, in: Zak O, Sande M, editors, Handbook of Animal Models of Infection, San Diego, Calif.: Academic Press (1999), at pages 389-403, the contents of which are incorporated herein by reference to illustrate the nature and acceptance in the art of this animal model.

To establish and evaluate the animal model, each chinchilla was inoculated with Haemophilus influenzae directly into the middle ear of each ear by transbullar injection at a concentration of 100 cfu in a volume of 0.2 mL. Each chinchilla was given an otoscope ear exam prior to being placed on study. Dosing with a composition of the invention or control oral amoxicillin began approximately 48 hours after the bacterial inoculation. All animals were administered Buprenorphine 0.05 mg/kg twice a day subcutaneously for analgesia for the duration of the study.

At the end of the dosing period (8 days after bacterial inoculation), each animal was euthanized, their ear canals washed with saline, and examined. In particular, samples from the middle ear from each chinchilla were collected. One ear sample was cultured overnight per laboratory procedures. Approximately 24 hours after the samples plated out, they were counted and the colony forming units (cfu) recorded.

### EXAMPLE 5

### Treatment of Otitis Media in Chinchilla Model (Liposomal Lipids)

The positive control article (amoxicillin) was administered orally by gavage to three chinchillas twice per day for 6 days, approximately 8 hours apart. 2, 4 or 6 drops of ofloxacin 0.3% liposome formulation or ciprofloxacin 0.3% liposome formulation were administered to two groups of three chinchillas each as a maximal feasible dose for these animals.

Results in each group (3 non-pregnant female animals/group) were as follows:
Untreated animals: Active infection still present after 6 days.
Control animals: Amoxicillin, 20 mg/kg BID for 6 days. No active infection present in any animal after 6 days.
Animals treated according to the methods of the invention:
   Ciprofloxacin, 4 drops BID for 6 days. No active infection in any animal after 6 days.
   Ofloxacin, 2 drops BID; 6 days. Active infection still present in two animals after 6 days; not present in the third animal.
   Ofloxacin, 4 drops BID; 6 days. Active infection still present in one animal after 6 days; not present in the other two animals.

### EXAMPLE 6

### Treatment of Otitis Media in a Chinchilla Model (Non-Liposomal Lipids)

Using formulations of ciprofloxacin in liposomes and non-liposomal lipids, following the protocol described in Example 5, the following results were obtained:

| | | | | Number ears |
|---|---|---|---|---|
| | | | | infected/total |
| Group | Formulation | | Treatment | number ears |
| 1 | None | | Untreated | 7 of 10 |
| 2 | Component(s) | Percentage(s) | Ciprofloxacin | 0 of 10 |
| | Phospholipon 90.H | 2.0 | Formulation 2 | |
| | Vit. E | 1.0 | (liposomal) | |
| | Ethyl alcohol 1% | 6.0 | | |
| | Propylene Glycol | 5.0 | | |
| | Ciprofoxacin HCL | 0.3 | | |
| | Benzethonium | 0.02 | | |
| | Chloride | | | |
| | Boric acid Powder | 1.6 | | |
| | Water, distilled | 84.08 | | |
| | | 100% | | |
| 3 | Component(s) | Percentage(s) | Ciprofloxacin | 0 of 10 |
| | Mineral Oil, light | 9.00 | Formulation 3 | |
| | Phospholipon 90H | 1.00 | (non-liposomal) | |
| | Span 60 | 2.00 | | |
| | Tween 60 | 1.00 | | |
| | Propylene Glycol | 5.00 | | |
| | Boric Acid | 1.90 | | |
| | H₂0, Distilled | 79.78 | | |
| | Benzethonium | 0.02 | | |
| | Chloride | | | |
| | Ciprofloxacin HCL | 0.3 | | |
| | | 100% | | |

These results demonstrate the efficacy of the present invention in treating middle ear infection in a relevant animal model, with dose dependency in the animals treated.

The invention illustratively described herein may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are set forth within the following claims.

In addition, where features or aspects of the invention are described in terms of Markush groups, those skilled in the art will recognize that the invention is also thereby described in terms of any individual member or subgroup of members of the Markush group.

That which is claimed is:

1. A method for treating or preventing a middle ear infection and sequelae thereof by transmembrane administration of a medicament thereto, said method comprising:
applying a transmembrane carrier composition to the outer surface of the tympanic membrane, said transmembrane carrier composition comprising a medicament useful in treating or preventing infections of the middle ear and sequelae thereof.

2. A method for treating or preventing a middle ear infection and sequelae thereof by transmembrane administration of a medicament thereto, said method comprising:
applying a non-sterically stabilized transmembrane carrier composition to the outer surface of the tympanic membrane, said transmembrane carrier composition comprising a medicament useful in treating or preventing infections of the middle ear and sequelae thereof.

3. The method according to claim 1, wherein the transmembrane carrier is a lipid vesicle.

4. The method according to claim 2, wherein the non-sterically stabilized transmembrane carrier is selected from the group of lipid vesicles consisting of liposomes, micelles, liosomes, niosomes and transfersomes.

5. The method according to claim 5, wherein the non-sterically stabilized transmembrane carrier is a liposome.

6. The method according to claims 1 or 2, wherein the transmembrane carrier composition is a liposome comprising:
a water soluble preservative, and
a lipid soluble anti-oxidant;
wherein at least 75% of the liposomes are from about 0.5 μm to about 10 μm in diameter; and
wherein said composition has a viscosity of at least 20,000 centipoise and contains less than 2% w/w of a viscosity enhancing agent.

7. The method according to claim 1, wherein said medicament is an antibiotic.

8. The method according to claim 2, wherein said medicament is an antibiotic.

9. The method according to claims 8 or 9, wherein the antibiotic is selected from the group consisting of quinolone antibiotics, penicillin antibiotics, macrolide antibiotics, cephalosporin antibiotics, sulfa antibiotics, and beta-lactamase inhibitors.

10. The method according to claims 8 or 9, wherein said antibiotic comprises ciprofloxacin, and is administered to treat or prevent a middle ear infection.

11. The method according to claims 8 or 9, wherein said antibiotic comprises ofloxacin, and is administered to treat or prevent a middle ear infection.

12. The method according to claims 8 or 9, wherein said antibiotic comprises sulfisoxazole, and is administered to treat or prevent a middle ear infection.

13. The method according to claims 8 or 9, wherein said antibiotic comprises amoxicillin, and is administered to treat or prevent a middle ear infection.

14. The method according to claims 8 or 9, wherein the antibiotic is provided in a concentration of 0.3% w/w of the composition.

15. The method according to claim 1, wherein said medicament is an anti-viral agent.

16. The method according to claim 2, wherein said medicament is an anti-viral agent.

17. The method according to claims 16 or 17, wherein the anti-viral agent is acyclovir.

18. The method according to claims 1 or 2, further comprising administration of a medicament to treat pain, infection or inflammation in the outer ear.

19. The method according to claim 19, wherein the medicament to treat pain, infection or inflammation in the outer ear is provided in a sterically stabilized lipid vesicle.

20. The method according to claim 20, wherein the sterically stabilized lipid vesicle is a liposome.

21. The method according to claim 22, wherein the liposome is stabilized with cholesterol.

22. The method according to claim 19, wherein said medicament is selected from the group consisting of celecoxib, naproxen, indomethacin, ketoprofen, glucosamine, methysulfonylmethane, pregnenolone, S-adenosylmethionene, and combinations of any two or more thereof.

23. The method according to claim 20, wherein said medicament is selected from the group consisting of celecoxib, naproxen, indomethacin, ketoprofen, glucosamine, methysulfonylmethane, pregnenolone, S-adenosylmethionene, and combinations of any two or more thereof.

24. The method according to claim 1 or claim 2, wherein the transmembrane carrier composition is applied to the tympanic membrane during an acute phase of middle ear infection.
